(19) United States
(12) Patent Application Publication
(10) Pub. No.: US 2014/0243933 A1
(43) Pub. Date: Aug. 28, 2014

(54) Modulation of the Activity of Mitochondria in Brown Adipose Tissue By Photobiomodulation for the Treatment of Obesity

(71) Applicant: Alec Ginggen, Plymouth, MA (US)

(72) Inventor: Alec Ginggen, Plymouth, MA (US)

(73) Assignee: Medos International Sarl, Le Locle (CH)

(21) Appl. No.: 13/775,564

(22) Filed: Feb. 25, 2013

(51) Int. Cl. A61N 5/06

(52) U.S. Cl. CPC A61N 5/0625
USPC 607/88

(57) ABSTRACT

A method for treating obesity by activating non-shivering thermogenesis in mitochondria of targeted brown adipose tissue. A photobiomodulation device including one or more light sources is provided. The mitochondria of the targeted brown adipose tissue is photobiomodulated by exposure to light produced by the light source at a wavelength that modulates photoactivity of an electron transport chain and increases proton transfer across a membrane of the mitochondria of the targeted brown adipose tissue towards an intern space of the mitochondria, without increasing production of adenosine triphosphate.

### BACKGROUND OF THE INVENTION

1. Field of the invention

The present invention relates to activation of brown adipose tissue. More particularly, the invention relates to modulation of activity of mitochondria in brown adipose tissue by photobiomodulation or low level light therapy, in particular, for the treatment of obesity.

2. Description of Related Art

Obesity is of primary concern in the current debate over the continuing costs of health care in this country. Overweight individuals are more susceptible to Many health risks such as, but not limited to, diabetes, cardiovascular disease, Alzheimers and certain cancers. This health issue is therefore of utmost importance with individuals of all ages. Bariatric surgery used to reduce obesity is limited to only relatively extreme cases due to the significant health risks associated therewith. It would therefore be more desirable to address the issue of obesity well in advance of the need for such extreme measures.

A basic underlying tenet in reducing obesity is that energy expended must exceed that which is taken in. Increasing cellular energy expenditure is a current area of research and development, as discussed in the publication entitled “Cellular Bioenergetics as a Target for Obesity Therapy” by Tseng et al., Nature Reviews Drug Discovery, Vol. 9 (June 2010) pp. 465-481, which is herein incorporated by reference in its entirety. Thermogenesis is the physiological process of heat production in organisms. Heat is produced during food digestion and absorption, exercise and in response to environmental changes, such as exposure to cold temperatures and change of diet. The production of heat resulting from exposure to cold temperatures and change in diet occurs primarily in the mitochondria of brown fat tissue (BAT). Imaging devices such as Positron Emission Tomography (PET) and Computer Tomography (CT) scans reveal that areas richest in BAT in adult individuals are concentrated primarily in the cervical-supraclavicular region, in a distinct fascial plane in the front of the neck, sometimes extending into the thoracic and lumbar region, as depicted in FIG. 3.

As discussed in the Tseng et al. publication, increased thermogenesis or cellular energy expenditure may be triggered by drugs. Of course, the use of drug treatment therapy has several drawbacks such as side effects and possible increased resistance when used over an extended period of time. Moreover, the use of drugs results in an increased production of Adenosine triphosphate (ATP), It is therefore desirable to promote thermogenesis in the treatment of obesity without the use of pharmacological agents and without increasing ATP production.

### SUMMARY OF THE INVENTION

An aspect of the present invention is to reduce obesity by modulation of the activity of mitochondria in brown adipose tissue using photobiomodulation (PBM) without increasing ATP production.

The present invention is directed to a method for treating, obesity by activating non-shivering thermogenesis in mitochondria of targeted brown adipose tissue. A photobiomodulation device including one or more light sources is provided. The mitochondria of the targeted brown adipose tissue is photobiomodulated by exposure to light. produced by the light source at a wavelength that modulates photoactivity of the electron transport chain and increases proton transfer across a membrane of the mitochondria of the targeted brown adipose tissue towards an intermembrane space of the mitochondria, without increasing production of ATP.

### BRIEF DESCRIPTION OF THE DRAWING

The foregoing and other features of the present invention will be more readily apparent from the following detailed description and drawings of illustrative embodiments of the invention wherein like reference numbers refer to similar elements throughout the several views and in which:

FIG. 1 is a schematic view of an external photobiomodulation device for the treatment of obesity in accordance with the present invention;

FIG. 2 is a schematic view of an implantable photobiomodulation device for the treatment of obesity in accordance with the present invention; and

FIG. 3 illustrates the richest areas of BAT depot in an adult human body;

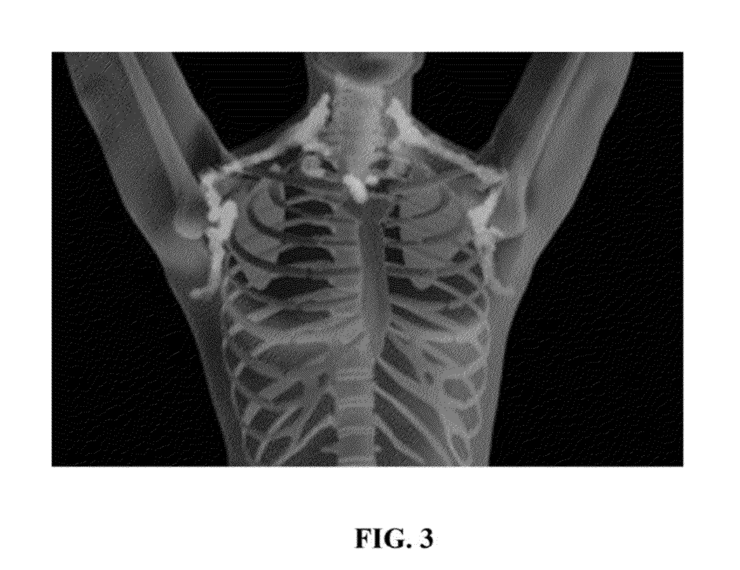

FIG. 4A is a partial view of a mitochondrial structure of BAT illustrating non-shivering thermogenesis in the absence of an external stimulus; and

FIG. 4B is a partial view of a mitochondrial structure of BAT illustrating non-shivering thermogenesis in the presence of PBM as an external stimulus.

### DETAILED DESCRIPTION OF THE INVENTION

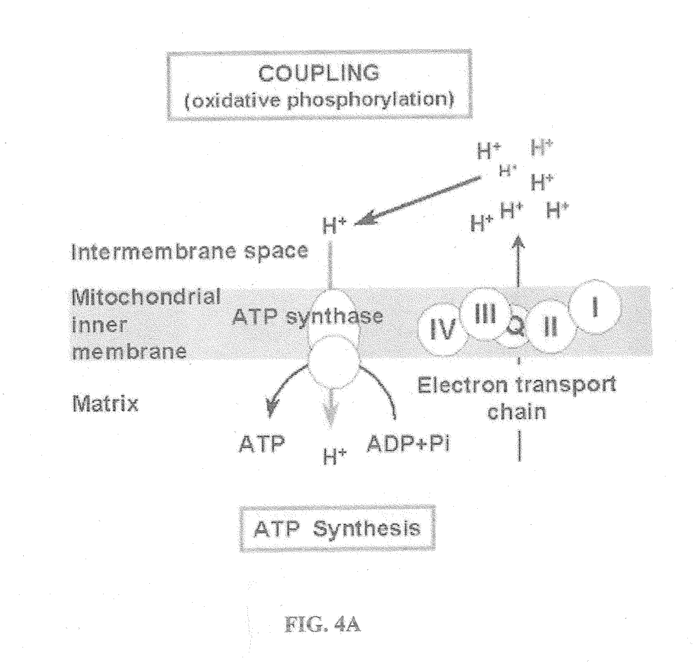

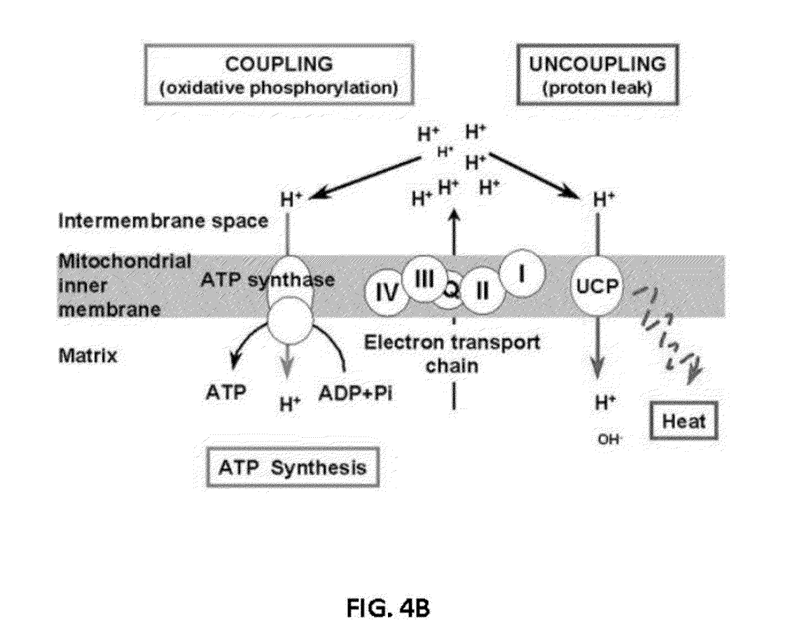

The physiological process of heat production in organisms, known as thermogenesis, may be classified as either shivering (activation of antagonistic muscle pairs) or non-shivering thermogenesis. Non-shivering thermogenesis occurs in the mitochondria of brown adipocytes (brown fat cells). FIG. 4A is a partial view of a mitochondrial structure of BAT (including the matrix; inner mitochondrial membrane and intermembrane space disposed between the inner and outer mitochondrial membranes) illustrating the process of non-shivering thermogenesis in the absence of an external stimulus. Nutrients and oxygen are converted by the mitochondria into released energy (heat) while water, carbon dioxide and Adenosine triphosphate (ATP) are produced. This conversion occurring within the mitochondria of BAT is part of the Krebbs cycle (also known as the “ticarboxylic acid cycle” or “TCA cycle”) and electron transport chain (ETC). In the absence of any external stimulus, this release of energy (heat) is triggered enzymatically as part of a series of reactions that optimize production of ATP while minimizing loss as heat. The generated ATP is distributed throughout the cell. Such chemical reactions may be classified as either coupled (resulting in some energy lost as heat) or uncoupled (energy consuming). Of particular interest in the present invention is the uncoupled reaction of the regulated proton leak in BAT by uncoupling protein-1 (UCP1) (thermogenin), which is an inner mitochondrial transmembrane protein proton transporter expressed only in brown adipocytes. Oxidative phosphorylation normally drives protons into the intermembrane space, generating an electrochemical gradient that pushes protons back into the mitochondrial matrix, activating ATP synthesis. The UCP1 protein allows protons in the mitochondrial intermembrane space to re-enter the inner mitochondrial membrane along their concentration gradient without generating ATP, that is, uncoupled. As a result, heat is generated directly by protons (H+ ions) rushing down their electrochemical gradient and also indirectly by the subsequent increase in flux through the ETC that follows. The uncoupling protein UCP1 is unique to BAT and necessary to mediate BAT thermogenesis. It is the presence of the trans-membrane protein UCP1 that allows the ETC and leakage of protons along their gradient.

In accordance with the present invention, mitochondria of BAT is exposed to an external stimulus, e.g., photobiomodulation (PBM), also known as low level light therapy (LLLT) that increases photo-activity of the ETC and increases the proton gradient across the mitochondrial membrane thereby increasing energy expenditure, without increasing the production of ATP that would otherwise result in the generation of heat. Accordingly, PBM results exclusively in increased energy consumption or expenditure, without any undesired heat byproduct generated as a result of increased ATP production. The light selected for PBM or LLLT is at a wavelength that modulates the activity of the ETC, and therefore, increases the proton transfer towards the intermembrane space of the mitochondria. Preferably, PBM or LLLT uses monochromatic light (typically red to infra-red light at a wavelength of approximately 600 nm to approximately 900 nm) at low intensity (i.e., negligible no thermal effect). Different light sources such as, but not limited to, one or more light emitting diodes (LEDs) or lasers may be employed to produce the low level light used in PBM. Referring to the right hand side of FIG. 4B, it is now contemplated and within the intended scope of the present invention to utilize PBM as an external stimulus to modulate activity of the mitochondria in brown adipose tissue (BAT) in the treatment of obesity. Exposure of the mitochondria in BAT to PBM boosts the ETC (denoted by the upwards arrow), and therefore, increases proton leakage (transfer) across the inner mitochondrial membrane towards the intermembrane space. Due to the presence of the UCP1 protein, the protons in the mitochondrial intermembrane space re-enter the inner mitochondrial space along their concentration gradient without generating ATP, that is, uncoupled. As denoted by the downward arrow on the right hand side of FIG. 4B, PBM increases heat generated directly by protons (H+ ions) rushing down their electrochemical gradient and also indirectly by the subsequent increase in flux through the ETC that follows.

Four major protein complexes Complex I, II, III, IV) localized within the inner mitochondrial membrane make up the ETC. Current understanding of the mechanism of action of PBM suggests that light is absorbed by cytochrome C oxidase (complex IV of the ETC) at about 680 nm and about 820 nm. These two wavelengths correspond to the excitation energy of the two copper centers of the COX enzyme and activate the ETC (see Kant, T. “Primary and secondary mechanisms of action of visible to near-IR radiation of cells,” I Photochem Photobiol B: Biol., Vol. 49 (1999) pp. 1-17, and Hayworth et al., “In vivo low-level light therapy increases cytochrome Oxidase in Skeletal Muscle”, Photochem, Photobiol. Vol. 86(2010) pp, 673-680, each of which is herein incorporated by reference in its entirety). The increased proton concentration gradient, in turn, increases leakage of protons along UCP1 and heat production. This leakage is un-coupled from ATP production through ATP synthase, thus heat is advantageously produced without increasing production of ATP.

The LED intensity is preferably in the range of approximately 0.5 mW/cm² to approximately 100 mW/cm² as measured at the level of the BAT cells (i.e., the intensity at the level of the skin is high as a result of absorption by soft tissue between the light emitter and the target BAT cells). The light therapy treatment in accordance with the present invention may be either continuous or pulsed. In the case of pulsed light, the preferred duration of each pulse is between approximately 1 second-approximately 60 minutes, with a preferred duration between consecutive pulses between approximately 100 seconds and approximately 24 hours. Treatment parameters (e.g., wavelength, intensity and/or duration) may be selected, as desired, so as to optimally activate the ETC by PBM with minimal, if any, generation of thermal effects (i.e., heating of the tissue).

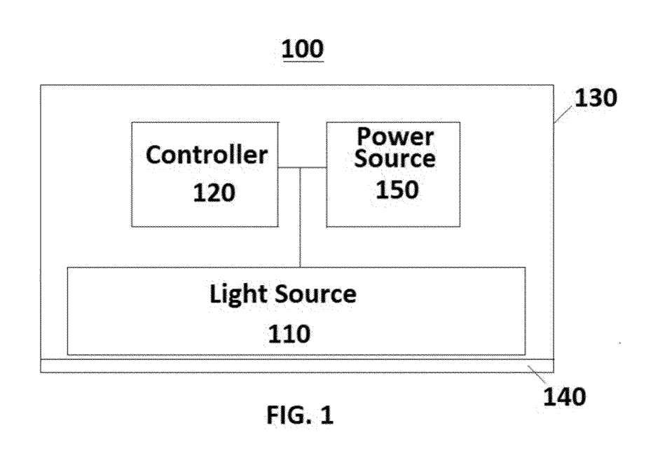

FIG. 1 is a schematic. diagram of a non-invasive external PBM device 100 for use in the treatment of Obesity in accordance with the present invention. Device 100 includes a power source 150 (e.g., battery) and one or more light sources 110, preferably one or more lasers, light emitting diodes (LEDs). Light source 110 produces light within at a specific wavelength or within a predetermined range of wavelengths that maximizes activation of the BAT mitochondria. Most preferably, due to its size and cost, an array of LEDs is a desirable light source. However, any conventional light source producing light in the specific wavelength to maximize activation of the BAT mitochondria may be utilized. The number, size, arrangement and placement of the one or more light sources may vary, as desired, depending on the size and location of the targeted BAT depot. Light source 110 produces light of a specific wavelength, intensity and/or duration to optimize activation of the ETC by PBM with negligible, if any, generation of thermal effects (i.e., heating of the tissue). Rather than a fixed light source, it is also contemplated to use a variable light source whose wavelength may be varied, as desired.

Preferably, the light generated by light source 110 is in the red (e.g., approximately 630 nm-approximately 740 nm) or infra-red (e.g., approximately 750 nm-approximately 900 mm) wavelength range since these wavelengths correspond to the excitation frequency of the Cytochrome C Oxydase (complex IV of the ETC) at about 680 nm and about 820 nm. Advantageously, soft tissue (e.g., skin, fat, muscle) is relatively transparent to light within these specified wavelength ranges. Light within the red or infrared frequency wavelength range generally has a penetration depth of approximately 1 cm-approximately 2 cm in soft tissue. Housing 130 of the device 100 has one or more windows or diffusers 140 through which the light produced by the source 110 is able to pass either completely unobstructed or only partially obstructed so that at least some light is able to pass therethrough. The diffusers homogenize the light intensity over the illuminated area. A single diffuser 140 in FIG. 1 spans the entire width of the PBM device 100, however, the size, shape, number and arrangement of the diffusers 140 may be modified, as desired.

A controller 120 such as a CPU, microprocessor or processor is also preferably included in the device 100 for varying, as desired, one or more control parameters associated with the light produced by the light source 110. By way of illustrative example, the one or more control parameters adjusted by the controller 120 may include at least one of intensity, wavelength, duration, pulse vs. continuous, selective activation/deactivation of one or more LED in the array, etc. The parameters associated with each of the LEDs may be controlled either independently or altogether as a group, by controller 120. All circuitry and components including controller 120 may be disposed within a single housing 130, as shown in FIG. 1, Alternatively, some of the circuitry and/or components may be disposed within a device separate from that of the light source. In such latter case, the two separate devices may communicate via a conventional wired or wireless communication interface.

In use, device 100 is positioned with one or more transparent diffusers 140 proximate the skin of the patient oriented so as to bathe in the generated light a targeted BAT depot, preferably a significant or predominant BAT depot, most preferably the BAT depot in the supra-clavicular region, in a distinct fascial plane in the front of the neck, sometimes extending into the thoracic and lumbar region. Light produced by the source 110 passes through the one or more diffusers 140 of the housing 130 as well as the soft tissue and bathing the targeted BAT depot. As previously noted, the light source 110 is selected to generate light at a particular wavelength and intensity to maximize activation of the targeted BAT mitochondria. The one or more light sources (e.g., LED) may be integrated in clothing or in a flexible substrate that conforms to at least a portion of the human body, e.g., the patient's shoulders. In an alternative embodiment, the one or more light sources may be an accessory/device to be worn, secured about, or supported by at least a portion of the patient's body. Power source 150 (e.g. battery) is used to power the controller 120, the one or more light sources 110 and all other electronic circuitry.

The external device in accordance with the first embodiment depicted in FIG. 1 is non-invasive. Despite employing a light source within the red or infrared spectra the generated light is only partially transparent to soft tissue (e.g., skin, fat, muscle). Thus, a certain amount of light will be impeded from reaching the targeted BAT mitochondria. When using an external device (as depicted in FIG. 1.), to insure photo-stimulation of the targeted BAT tissue, an area or region of the body larger than the targeted BAT depot is typically exposed to the relatively low level light resulting in possible exposure of non-targeted tissue.

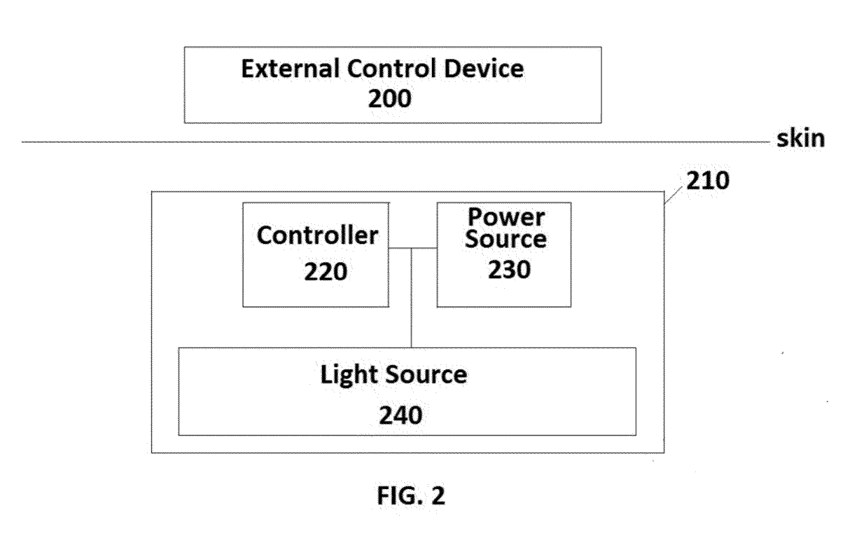

The aforementioned disadvantages may be overcome by using an implantable PBM device 210, as illustrated in FIG. 2. Rather than being applied external to the body, the device 210 including the light source 240 is implantable proximate the targeted BAT depot (e.g., supra-clavicular region) and may be controlled by way of an external control device 200. Implantation of the PBM device 210 allows for more precise targeting of BAT depot without effecting surrounding tissue. In addition, since the light generated by the light source need not pass through soft tissue (e.g., skin, fat, muscle) before arriving at the targeted BAT depot, light sources having a wide spectrum of wavelength frequencies may be employed with the implantable PMB device. All the advantages associated with the implantable PMB device, of course, must be balanced against opposing increased costs and health risks associated with any type of surgical implantation procedure.

Referring to FIG. 2, the PBM system includes a PBM device 210 implanted proximate the targeted BAT depot which, in turn, is controlled by an external control device 200. Wireless communication between the external control device and implantable PBM device 210 may occur using any conventional wired or preferably wireless communication interface. As with the external PBM device, the implantable PBM device 210 includes one or more light sources 240 energized by an internal power source 230 (e.g., battery) and controlled by a controller 220 (e.g., a microprocessor, processor, CPU. etc.).

Photobiomodulation modulates the activity of the mitochondria by photo-activating complex IV of the ETC, thereby increasing proton transport towards the intermembrane space without increasing ATP production. It is hypothesized that complex IV in the ETC (cytochrome C oxydase) acts as a photon acceptor. The accepted photon displaces nitric oxide bound to complex IV, allowing oxygen binding and activation of the proton transport. As a result, the proton gradient across the membrane increases and, in turn, the leakage of proton through UCP1 increases to re-establish a lower concentration gradient.

Exposure of BAT mitochondria to PBM has the following effects: (i) increased activity of the ETC; (ii) increased proton leakage along an electro-chemical gradient due to the presence of UCP; (iii) increased proton gradient across the mitochondria(membrane; (iv) increased heat generation; (v) increased energy expenditure; and (vi) optimization of balance between energy intake and energy expenditure in obese patients. All these effects occur simultaneously without increasing ATP production.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also filly intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A method for treating obesity by activating non-shivering thermogenesis in mitochondria of targeted brown adipose tissue, comprising the steps of;
providing a photobiomodulation device including at least one light source; and
photobiomodulating the mitochondria of the targeted brown adipose tissue by exposure to light produced by the at least one light source at a wavelength that modulates photoactivity of an electron transport chain and increases proton transfer across a membrane of the mitochondria of the targeted brown adipose tissue towards an intermembrane space of the mitochondria.

2. The method in accordance with claim 1, wherein the step of photobiomodulating the mitochondria of the targeted brown adipose tissue does not increase production of adenosine triphosphate.

3. The method in accordance with claim 1, wherein the at least one light source is a monochromatic light source in a red to infrared light spectra.

4. The method in accordance with claim 3, wherein the wavelength of the at least one light source is constant.

5. The method in accordance with claim 3, wherein the wavelength of the at least one light source is variable.

6. The method in accordance with claim 3, wherein the wavelength is approximately 600 nm to approximately 900 nm.

7. The method in accordance with claim 6, wherein the wavelength is approximately 680 nm or approximately 820 nm.

8. The method in accordance with claim 3, wherein intensity of the at least one light source is in the range of approximately 0.5 mW/cm² to approximately 100 mW/cm² as measured at the targeted brown adipose tissue.

9. The method in accordance with claim 1, wherein the light is continuous.

10. The method in accordance with claim 1, wherein the light is pulsed.

11. The method in accordance with claim 10, wherein the light is pulsed, each pulse being, between approximately 1 second-approximately 60 minutes in duration; and a time period between consecutive pulses being between approximately 100 seconds and approximately 24 hours.

12. The method in accordance with claim 1, wherein the photobiomodulation device is positioned externally proximate the targeted brown adipose tissue.

13. The method in accordance with claim 1, wherein the photobiomodulation device is implanted proximate the targeted brown adipose tissue.

14. The method in accordance with claim 1, wherein the proton transfer across a membrane of the mitochondria of the targeted brown adipose tissue is uncoupled from adenosine triphosphate production through adenosine triphosphate synthase so that heat is produced without increasing production of adenosine triphosphate.

15. The method in accordance with claim 1, wherein in the photobiomodulating step heat is generated directly by protons rushing down their electrochemical gradient and also indirectly by a subsequent increase in flux through the electron transport chain that follows, without producing adenosine triphosphate.

16. The method in accordance with claim 15, wherein the protons are H+ ions.

17. The method in accordance with claim 1, further comprising the step of adjusting via a controller at least one parameter associated with the light, produced by the at least one light source.

18. The method in accordance with claim 17, wherein the at least one parameter includes at least one of intensity, wavelength, duration, pulsed versus continuous, selective activation/deactivation of the at least one light source.

19. The method in accordance with claim 18, wherein the at least one parameter is independently selectable for each of the at least one light sources.

20. The method in accordance with claim 18, wherein the at least one parameter is selectable for the at least one light sources together as a group.

21. The method in accordance with claim 1, wherein photobiomodulating step comprises photo-activating complex IV of the electron transport chain.
